SEQUENCE LISTING (15 sequences)

SEQ ID NO: 1 - DNA, 34 bp, Artificial
  Synthetic DNA
ataacttcgtatagcatacattatacgaagttat

SEQ ID NO: 2 - DNA, 34 bp, Artificial
  Synthetic DNA
taccgttcgtatagcatacattatacgaagttat

SEQ ID NO: 3 - DNA, 34 bp, Artificial
  Synthetic DNA
ataacttcgtatagcatacattatacgaacggta

SEQ ID NO: 4 - DNA, 34 bp, Artificial
  Synthetic DNA
ataacttcgtatagtatacattatacgaagttat

SEQ ID NO: 5 - DNA, 34 bp, Artificial
  Synthetic DNA
ataacttcgtataggatactttatacgaagttat

SEQ ID NO: 6 - DNA, 34 bp, Artificial
  Synthetic DNA
tcaatttctgagaactgtcattctcggaaattga

SEQ ID NO: 7 - DNA, 34 bp, Artificial
  Synthetic DNA
cgtgattctgagaactgtcattctcggaaattga

SEQ ID NO: 8 - DNA, 34 bp, Artificial
  Synthetic DNA
tcaatttctgagaactgtcattctcggaatacct

SEQ ID NO: 9 - DNA, 34 bp, Artificial
  Synthetic DNA
tcaatttctgagaagtgtctttctcggaaattga

SEQ ID NO: 10 - DNA, 32 bp, Artificial
  Synthetic DNA
taactttaaataatgccaattatttaaagtta

SEQ ID NO: 11 - DNA, 32 bp, Artificial
  Synthetic DNA
taactttaaataattggcattatttaaagtta

SEQ ID NO: 12 - DNA, 34 bp, Artificial
  Synthetic DNA
ataacttcgtataatgtatgctatacgaagttat

SEQ ID NO: 13 - DNA, 34 bp, Artificial; identical to SEQ ID NO: 1
  Synthetic DNA

SEQ ID NO: 14 - DNA, 21 bp, Artificial
  Synthetic DNA
ccatggcccccaagaagaaaa

SEQ ID NO: 15 - DNA, 22 bp, Artificial
  Synthetic DNA
cgggatccatgagcctgctgtt

(19) United States
(12) Patent Application Publication
(10) Pub. No.: US 2018/0360006 A1
(43) Pub. Date: Dec. 20, 2018

(54) ORGAN HUMANIZED MOUSE

(71) Applicant: TRANSGENIC INC., Fukuoka-shi, Fukuoka (JP)

(72) Inventors: Kenichi YAMAMURA, Hyogo (JP); Zhenghua LI, Hyogo (JP)

(73) Assignee: TRANSGENIC INC., Fukuoka-shi, Fukuoka (JP)

(21) Appl. No.: 15/736,899

(22) PCT Filed: Jun. 18, 2015
(86) PCT No.: PCT/JP2015/067541

§ 371 Date: Dec. 15, 2017

(51) Int. Cl. A01K 67/027; C12N 5/0735

(52) U.S. Cl. CPC A01K 67/0278; C12N 5/0606; A01K 2217/075; A01K 2267/03; A01K 2227/105; C12N 2501/998

(57) ABSTRACT

The present invention provides embryonic stem cells obtained from an embryo of a mouse engineered to replace all or some of domains in the mouse MHC class I molecule H2-D with domains from the human MHC class I molecule HLA-A by culture in the presence of a GSK3 inhibitor and an MEK inhibitor, as well as a mouse which is created with the use of these embryonic stem cells.

### TECHNICAL FIELD

The present invention relates to embryonic stem cells (ES cells) taken from a mouse engineered to replace all or some of domains of the mouse MHC class I molecule H2-D with domains from the human MHC class I molecule HLA-A as well as a mouse with a humanized organ.

### BACKGROUND ART

Among previous reports on the preparation of human organ model mice, as an example of liver model mice, Heckel et al. have reported transgenic mice (Tg(Alb-Plau)) carrying a construct (Alb-Plau) composed of the urokinase-type plasminogen activator (Plau) gene linked to the albumin (Alb) promoter (Non-patent Document 1: Heckel et al. Cell 62:447-456, 1990)). However, these original mice cannot be used for experiments because they will die within 4 days after birth due to hemorrhage in their intestinal tract and elsewhere. On the other hand, the same research group has succeeded in establishing lines of survivors from among Tg(Alb-Plau) mice and has reported a case where the liver was regenerated from liver cells which were deficient in the Alb-Plau gene during liver cell division (Non-patent Document 2: Sandgren et al. Cell 66:245-256, 1991). Moreover, there is a report showing successful transplantation of Tg(Alb-Plau) with mature liver cells from a transgenic mouse (Tg(MT-nLacZ) mouse) carrying the lacZ gene linked to the metallothionein promoter, i.e., a mouse whose liver cells serving as a donor were labeled with the marker gene lacZ (Non-patent Document 3: Rhim et al. Science 263:1149-1152, 1994).

In addition, there are reports on the transplantation of immunodeficient mice with human liver cells, as exemplified by a report in which Rag2 deficient immunodeficient mice were transplanted with liver cells, followed by infection experiment with hepatitis B virus (HBV) (Non-patent Document 4: Dandri et al. Hepatology 33:981-988, 2001), or a report in which Tg(Alb-Plau) mice were crossed with SCID mice, which are immunodeficient mice, and the resulting immunodeficient SCID mice (Tg(Alb-Plau)) were then transplanted with human liver cells (Tg(Alb-Plau); SCID)), followed by infection experiment with hepatitis C virus (Non-patent Document 5: Mercer et al. Nature Med. 7:927-933, 2001).

Further, Tateno et al. have reported that albumin enhancer/promoter urokinase plasminogen activator transgenic mice (uPA mice) undergoing liver failure were crossed with SCID mice to prepare uPA/SCID transgenic mice homozygouse for both loci (Non-patent Document 6: Tateno et al. Amer. J. Pathol 165:901-912, 2004). This report discusses improved techniques for transplantation of human liver cells into Tg(Alb-Plau; SCID), in which Futhan treatment is used to eliminate the effects of complements derived from human liver cells to thereby reduce the mortality even at high chimerism.

Moreover, there is a report on the study which demonstrates the possibility of Rag2-deficient immunodeficient mice as a model for gene therapy (Non-patent Document 7: Orthopedic Surgery and Traumatology “Series IV of Orthopedic Diseases from the Molecular Level, Somatic Cell Cloning Technology and Regenerative Medicine” Vol. 45, NO. 11, PAGE. 1040-1041, 2002 (in Japanese)).

However, these model mice do not serve as a liver cell model in which 100% of the cells are replaced with cells of human origin, because host mouse liver cells are left therein. In addition, cells of human origin do not always regenerate, so that cells of human origin should be transplanted. Moreover, when liver cells of mouse origin are left, human liver functions cannot be verified sufficiently.

On the other hand, for establishment of NOG mouse-derived ES cell lines for germ-line transmission, some attempts have also been made to establish ES cells by using differentiation signal inhibitors (PD0325901, CHIR99021) (Non-patent Document 8: Abstracts of the Annual Meeting of the Japanese Association for Laboratory Animal Science, Vol. 58th, Page 210, 2011 (in Japanese)).

However, NOG mice are difficult to obtain in large number for use in experiments because they are difficult to breed.

In addition, the inventors of the present invention have previously succeeded in creating an animal model with a humanized liver, starting from embryonic stem cells taken from an immunodeficient mouse (Patent Document 1: WO2013/145331).

### PRIOR ART DOCUMENTS

### Patent Documents

Patent Document 1: WO2013/145331

### Non-Patent Documents

Non-patent Document 1: Heckel et al. Cell 62:447-456, 1990

Non-patent Document 2: Sandgren et al. Cell 66:245-256, 1991

Non-patent Document 3: Rhim et al. Science 263:1149-1152, 1994

Non-patent Document 4: Dandri et al. Hepatology 33:981-988, 2001

Non-patent Document 5: Mercer et al. Nature Med. 7:927-933, 2001

Non-patent Document 6: Tateno et al. Amer. J. Pathol 165:901-912, 2004

Non-patent Document 7: Orthopedic Surgery and Traumatology “Series IV of Orthopedic Diseases from the Molecular Level, Somatic Cell Cloning Technology and Regenerative Medicine” Vol. 45, NO. 11, PAGE. 1040-1041, 2002 (in Japanese)

Non-patent Document 8: Abstracts of the Annual Meeting of the Japanese Association for Laboratory Animal Science, Vol. 58th, Page 210, 2011 (in Japanese)

### SUMMARY OF THE INVENTION

### Problem to be Solved by the Invention

The present invention aims to provide a mouse with a humanized organ, starting from an embryo derived from a mouse showing normal immune responses, but not from an immunodeficient mouse. More specifically, the present invention aims to provide embryonic stem cells (ES cells) taken from a mouse engineered to disrupt its mouse major histocompatibility antigen (MHC) class I gene and instead have the human major histocompatibility antigen class I gene, as well as a mouse with a humanized organ.

### Means to Solve the Problem

As a result of extensive and intensive efforts made to solve the problems stated above, the inventors of the present invention have succeeded in creating a mouse with a humanized organ from embryonic stem cells obtained by preparing an embryo of a mouse engineered to replace all or some of domains in the mouse MHC class I molecule H2-D with domains from the human MHC class I molecule HLA-A, and culturing this embryo in the presence of a GSK3 inhibitor and an MEK inhibitor.

The inventors of the present invention have found that when human liver cells are transplanted into the yolk sac vessel of this mouse at its fetal stage, the human liver cells can be engrafted without the necessity to use any immunodeficient mouse as in conventional cases. This finding led to the completion of the present invention.

Namely, the present invention is as follows.
(1) An embryonic stem cell obtained from an embryo of a mouse engineered to replace all or some of domains in the mouse MHC class I molecule H2-D with domains from the human MHC class I molecule HLA-A by culture in the presence of a GSK3 inhibitor and an MEK inhibitor.
(2) The embryonic stem cell according to (1) above, wherein the α1 domain, α2 domain of the H2-D molecule and β2 microglobulin are replaced with the α1 domain, α2 domain of the human HLA-A molecule and β2 microglobulin, respectively.
(3) The embryonic stem cell according to (1) or (2) above, which is deposited under Accession No. NITE ABP-02068.
(4) The embryonic stem cell according to any one of (1) to (3) above, which is engineered to have the estrogen receptor gene and the diphtheria toxin gene.
(5) The embryonic stem cell according to (4) above, wherein the endogenous growth hormone gene in the cell is replaced with that of human origin.
(6) The embryonic stem cell according to (5) above, wherein an endogenous drug-metabolizing enzyme gene in the cell is further replaced with that of human origin.
(7) The embryonic stem cell according to (6) above, wherein the endogenous drug-metabolizing enzyme gene in the cell is at least one selected from the group consisting of Cyp3a11, Cyp3a13, Cyp3a25 and Cyp3a41.
(8) A mouse, which is created with the use of the embryonic stem cell according to any one of (1) to (3) above.
(9) A mouse, which is created with the use of the embryonic stem cell according to any one of (4) to (7) above.
(10) The mouse according to (9) above, which develops liver cell injury upon administration of an antiestrogen.
(11) A mouse with a humanized liver, wherein the mouse according to (9) above is transplanted with liver cells of human origin and also administered with an antiestrogen to eliminate liver cells originating from the mouse.
(12) The mouse according to (11) above, wherein the liver cells of human origin are derived from a patient with a liver disease.
(13) A human liver disease model mouse, which consists of the mouse according to (12) above.
(14) A method for preparing an embryonic stem cell of mouse origin, which comprises culturing, in the presence of a GSK3 inhibitor and an MEK inhibitor, an embryo of a mouse engineered to replace all or some of domains of the mouse MHC class I molecule H2-D with domains from the human MHC class I molecule HLA-A.
(15) The method according to (14) above, wherein the α1 domain, α2 domain of the H2-D molecule and β2 microglobulin are replaced with the α1 domain, α2 domain of the human HLA-A molecule and β2 microglobulin, respectively.
(16) A method for creating a liver injury model mouse, which comprises administering an antiestrogen to the mouse according to (9) above.
(17) A method for creating a mouse with a humanized liver, which comprises transplanting liver cells of human origin into the mouse according to (9) above and also administering an antiestrogen to eliminate liver cells originating from the mouse.
(18) The method according to (17) above, wherein the liver cells of human origin are derived from a patient with a liver disease.

### Effects of the Invention

The present invention provides embryonic stem cells which are derived from mouse showing normal immune responses and are used for establishment of a mouse most suitable for human cell transplantation. For liver humanization, the embryonic stem cells of the present invention can be engineered to have various human genes related to liver functions to thereby establish a humanized liver model mouse. Thus, a mouse established from the embryonic stem cells of the present invention is very useful in that it can be used for transplantation of cells from various human organs and achieves 100% humanization.

### BRIEF DESCRIPTION OF THE DRAWINGS

FIG. 1 shows an MHC class I molecule used for creation of an HHB mouse.

FIG. 2 shows various lox mutants.

FIG. 3 shows a scheme for construction of a replacement vector used for introduction of the human growth hormone gene into ES cells.

FIG. 4 shows a scheme for construction of a replacement vector used for introduction of a human drug-metabolizing enzyme gene into ES cells.

FIG. 5 shows a scheme for the process starting from introduction of the diphtheria toxin gene into ES cells until cell death in mouse liver cells.

FIG. 6 shows the site for transplantation of human liver cells into a mouse embryo.

FIG. 7 shows the process of inducing differentiation from iPS cells into liver cells.

### DESCRIPTION OF EMBODIMENTS

The present invention will be described in more detail below.

### 1. Summary

The present invention has been made to provide embryonic stem cells established from an embryo of a mouse engineered to replace all or some of domains of the mouse MHC class I molecule H2-D in a normal mouse with domains from the human MHC class I molecule HLA-A, and further has been made to establish a mouse with a humanized organ from these embryonic stem cells.

In general, when a mouse fetus is transplanted with human cells, these cells are engrafted in the mouse body, so that the resulting mouse is humanized at the cellular level.

However, in such a humanized mouse, cells originating from the host mouse are left therein, and hence not all of its organs are replaced with those of human origin. For this reason, such a humanized mouse is not necessarily optimized for functional analysis or study on these organs. Moreover, various genetic modifications are required to prepare an optimized mouse, although a whole mouse cannot be used for this purpose.

Thus, for establishment of a mouse with a liver whose cells have all been humanized, the present invention aims to establish a genetically modified mouse which is most suitable for humanization. As a result of extensive and intensive efforts aimed at humanization from the early stage of ontogeny in this genetically modified mouse, the inventors of the present invention have succeeded in establishing embryonic stem cells (hereinafter referred to as “ES cells”) from normal and HHB mice. The inventors of the present invention have also succeeded in preparing a germ-line chimeric mouse using the ES cells.

In the present invention, the inventors have succeeded in establishing desired ES cells from HHB mice, and have also succeeded in establishing desired ES cells from normal mice, as described later.

The mouse of the present invention has been engineered to replace all or some of domains of the mouse MHC class I molecule H2-D with domains from the human MHC class I molecule HLA-A.

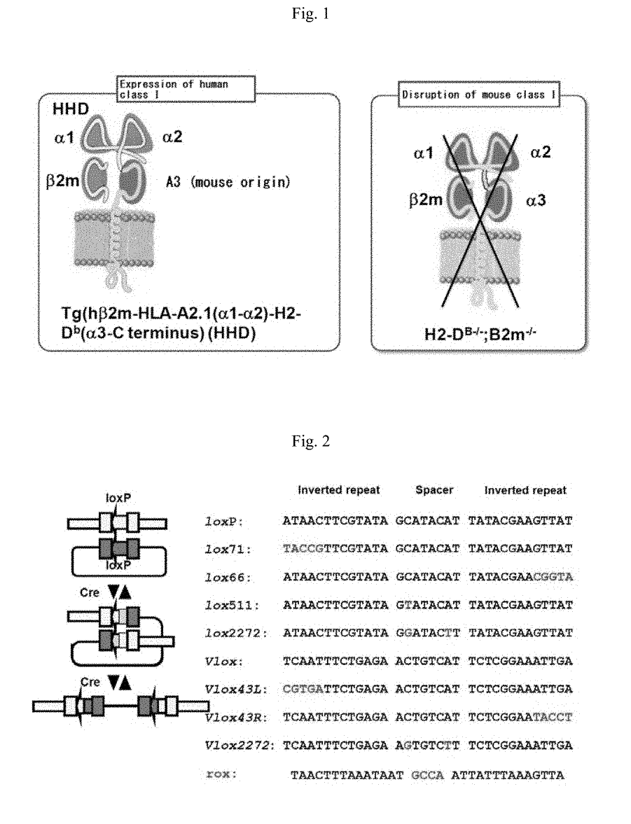

An MHC class I molecule is a dimer composed of an a chain (heavy chain) and a β2-microglobulin chain (light chain), which are non-covalently bound to each other, and is expressed on the cell surface as a trimer together with a peptide antigen further bound thereto. The α chain molecule is composed of the following domains, i.e., three extracellular regions α1 to α3, a transmembrane region and an intracellular region. Domains (chains) to be replaced between mouse and human molecules are the α1 and α2 chains of α chain and the β2-microglobulin chain. In the present invention, all of the domains can be replaced with those of human origin, but it is preferred that the α3 domain is of mouse origin and the domains other than α3 are all replaced with those of human origin (FIG. 1).

In a preferred embodiment of the present invention, the mouse of the present invention is a mouse engineered not only to disrupt the mouse class H2-D gene and the mouse β2-microglobulin gene, but also to have genes for α1 and α2 domains of human HLA-A2.1. In this embodiment, only the α3 domain is of mouse origin. This mouse is designated as “HHB mouse.”

In addition, for maintenance of liver functions over a long period of time and for confirmation of the safety, the present invention aims to establish a mouse with a human normal liver. Moreover, for establishment of a disease model having the same symptoms as seen in human patients with liver disease and for analysis of the pathology, the present invention aims to establish a mouse with a human mutated liver. Furthermore, for development of a novel therapy used for a wide range of purposes, the present invention aims to establish a model mouse optimized for human diseases.

2. Preparation of Mouse

The mouse of the present invention is created with the use of an embryo of a mouse engineered to replace all or some of domains of the mouse MHC class I molecule H2-D with domains from the human MHC class I molecule HLA-A.

In this mouse, the H2-D and β2-microglobulin genes have both been knocked out and further replaced with the human HLA-A2.1 gene. Preferably, the α1 and α2 domains are encoded by genes of human origin, while the α3 domain is encoded by a gene of mouse origin (FIG. 1). A gene encoding the molecule shown in FIG. 1 (left panel) is referred to as the “HHD gene” and a mouse having the HHD gene is referred to as “HHB mouse.” Such an HHB mouse has already been established (Pascolo, S., Bervas, N., Ure, J. M., Smith, A. G., Lemonnier, F. A. and Perarnau, B. HLA-A2.1-restricted education and cytolytic activity of CD8+ T lymphocytes from b2 microglobulin (b2m) HLA-A2.1 monochain transgenic H-2Db b2m double knockout mice. J. Exp. Med. 185:2043-2051, 1997).

How to prepare this mouse whose H2-D and β2-microglobulin genes have been knocked out and details on these genes are described in Pacolo et al. J. Exp. Med. 185:2043-2051, 1997, although such a mouse can be prepared by any technique well known in the art, e.g., the technique using a targeting vector (Capecchi, M. R., Science, (1989) 244, 1288-1292). This technique is based on homologous recombination between the H2-D or β2-microglobulin gene in mouse ES cells and a gene on the targeting vector.

It should be noted that HHB mice are also available from the Institute of Resource Development and Analysis, Kumamoto University, Japan. These mice can be back-crossed with commercially available C57BL/6 mice to thereby obtain a H2-D-deficient (−/−) mouse and a β2-microglobulin-deficient (−/−) mouse, each having the same genetic background as C57BL/6 mice.

To prepare a double knockout mouse deficient in both H2-D and β2-microglobulin genes, the C57BL/6-H2-D-deficient mouse and the C57BL/6-β2-microglobulin-deficient mouse are first crossed with each other to obtain F1 mice, followed by crossing between F1 mice to obtain F2 mice. From among these mice, a double-deficient, i.e., H2-D-deficient (−/−) and β2-microglobulin-deficient (−/−) mouse (C57BL/6-H2-D^{-/-}:β2-microglobulin^{-/-} mouse) may then be selected. As to techniques for selection of a C57BL/6-H2-D^{-/-}:β2-microglobulin^{-/-} mouse, for example, deficiencies in both H2-D and β2-microglobulin genes can be confirmed by PCR or Southern blotting.

Further, how to inject the HHD gene into a mouse fertilized egg to thereby obtain a transgenic mouse (Tg(HHD) mouse) is described in Pacolo et al. J. Exp. Med. 185:2043-2051, 1997. This mouse may be crossed with the C57BL/6-H2-D^{-/-}:β2-microglobulin^{-/-} mouse to obtain a C57BL/6-H2-D^{-/-}:β2-microglobulin^{-/-}:Tg(HHD) mouse (i.e., HHB mouse) (FIG. 1).

In addition to the HHD gene, all domains in the mouse H2-D molecule may be replaced with those of human origin, or all domains except for the α3 domain may be of mouse origin. Genes encoding such domains can be obtained by standard genetic engineering techniques.

3. Establishment of ES Cells

The ES cells of the present invention can be obtained from embryos taken from mice obtained as above by culture in the presence of a GSK3 inhibitor and an MEK inhibitor.

For example, in the case of using HHB mice, fertilized eggs or two-cell embryos are first obtained by being cultured or blastocysts are directly obtained from female HHB mice after fertilization. Fertilization may be accomplished by natural crossing or in vitro fertilization techniques. In the case of in vitro fertilization, ova obtained by superovulation of female mice and sperm taken from male mice may be cultured together.

Then, the collected blastocysts or inner cell mass may be cultured in a medium for animal cell culture in the presence of a GSK-3 inhibitor and an MEK inhibitor for about 1 to 3 weeks, preferably 14 to 18 days.

GSK-3 (glycogen synthase kinase 3), which is a serine/threonine protein kinase, is an enzyme acting on many signaling pathways responsible for glycogen production, apoptosis, stem cell maintenance and other events. Examples of a GSK-3 inhibitor include CHIR99021 (available from Wako Pure Chemical Industries, Ltd., Japan), 6-bromoindirubin-3′-oxime (BIO) (available from Wako Pure Chemical Industries, Ltd., Japan) and so on. Such a GSK-3 inhibitor may be added to the medium in an amount of 0.1 to 10 μM (micromolar), preferably 0.3 to 3 μM. The timing of GSK-3 inhibitor addition to the medium is not limited in any way, but it is preferably added from the beginning of blastocyst culture.

An MEK inhibitor is a protein kinase inhibitor which inhibits MAP Kinase Kinase (MEK) activity and suppresses ERK1/ERK2 activation. Examples of an MEK inhibitor include PD0325901 (available from Wako Pure Chemical Industries, Ltd., Japan), U0126 (available from Promega) and so on. The PD0325901 inhibitor may be added to the medium in any amount, for example, 3 μM.

Culture may be accomplished under any conditions, for example, at 37° C. in a 5% CO₂ atmosphere. Subculture may be conducted at an interval of 3 to 4 days on mouse embryo fibroblast (MEF) feeders or on collagenase I-coated plates.

Examples of the above medium include GMEM medium (Glasgow's Minimal Essential Medium), DMEM (Dulbecco's Modified Eagle's Medium), RPMI 1640 medium and so on. The culture medium may be supplemented as appropriate with an additional ingredient(s) selected from KSR (knockout serum replacement), fetal bovine serum (FBS), basic fibroblast growth factor (bFGF), β-mercaptoethanol, nonessential amino acids, glutamic acid, sodium pyruvate and antibiotics (e.g., penicillin, streptomycin), etc.

Culture may be continued for a given period of time, followed by incubation in a medium containing EDTA or collagenase IV to collect ES cells. The collected ES cells may optionally be subcultured several times by culture in the presence or absence of feeder cells. It should be noted that inner cell mass culture under feeder-free conditions may be conducted in an MEF-conditioned medium.

The cultured ES cells may usually be identified using their marker genes. Examples of marker genes in ES cells include Oct3/4, alkaline phosphatase, Sox2, Nanog, GDF3, REX1, FGF4 and so on. The presence of marker genes or gene products may be detected by any technique such as PCR or Western blotting.

Moreover, to determine whether or not the ES cells of the present invention are obtained as desired, whether they are of BALB/c origin can be confirmed by SNP marker detection, or by PCR or Southern blotting analysis. For example, a database of mouse SNPs is published at http://www.broadinstitute.org/snp/mouse, and when SNP information is compared against this database, the ES cells can be confirmed to be of BALB/c origin, so that they are determined to be the ES cells of the present invention.

The thus obtained ES cells were designated as “HHB10” and internationally deposited under the Budapest Treaty on Jun. 17, 2015 (receipt date) with the National Institute of Technology and Evaluation, Patent Microorganisms Depositary (2-5-8 Kazusakamatari, Kisarazu-shi, Chiba 292-0818, Japan). Their Accession No. is “NITE ABP-02068.”

Detailed information on the above ES cells is as follows.

Articles have already been published about how to establish ES cells with a GSK inhibitor and an MEK inhibitor, and the resulting ES cells inherit genetic characters of their original lines and contain their respective unique features.

In preliminary experiments, attempts were made to establish ES cells in the conventionally used GMEM-KSR medium, but only a strain showing very poor growth could be established. Even when used to prepare a chimeric mouse, this strain resulted in chimerism as low as 50% and did not contribute to the germ line. In contrast, in the present invention, a GSK3 inhibitor and an MEK inhibitor, which are considered to be effective for maintenance of the undifferentiated state of ES cells, were added to the medium to thereby achieve the establishment of the desired ES cells. The ES cells of the present invention are high in viability and also high in chimerism. This is because the ES cells of the present invention successfully maintain their undifferentiated state in comparison with ES cells prepared by conventional techniques. One of the important signals responsible for differentiation of ES cells is the ERK/MEK pathway from FGF4 through FGF receptors. Namely, ERK acts as a differentiation signal. On the other hand, GSK-3 stimulates Wnt signals through phosphorylation of β-catenin to thereby induce differentiation. Thus, by using two inhibitors (2i), i.e., a strong MEK inhibitor (PD0325901) and a GSK3 inhibitor, the ES cells of the present invention can be prevented from differentiation and hence maintain their pluripotency.

4. Genetic Modifications

To establish a genetically modified mouse which is most suitable for humanization, endogenous genes should be replaced with those of human origin at the stage of ES cells, but not in adult mice, or ES cells should be transformed with human genes, followed by creation of a mouse from the thus genetically modified and/or transformed ES cells.

Thus, in the present invention, for transformation of ES cells with desired genes or for replacement of endogenous genes in ES cells with human genes, homologous recombination with the following systems may be used: the bacteriophage-derived recombination system Cre-loxP, the Vibrio sp.-derived recombination system VCre-Vlox, the Cre homolog-mediated recombination system Dre/rox, or any system modified from these recombination systems.

loxP (locus of crossing (X-ring) over, P1) is a sequence of 34 nucleotides (5′-ATAACTTCGTATA GCATACAT TATACGAAGTTAT-3′) (SEQ ID NO: 1), in which sequences of the 5′-terminal 13 nucleotides (referred to as inverted repeat 1) and the 3′-terminal 13 nucleotides (referred to as inverted repeat 2) each constitute an inverted repeat, and a sequence represented by “GCATACAT” which is called a 8-nucleotide spacer is sandwiched between the above inverted repeats 1 and 2 (FIG. 2). The term “inverted repeat” is intended to mean a sequence, one of whose terminal segments is complementary to the other when they are opposed to each other via a spacer serving as their boundary.

Cre (causes recombination) is intended to mean a recombination enzyme (also referred to as a recombinase) which causes gene recombination, and it recognizes the above repeats to cleave the spacer in such a cleavage fashion that “cataca” in the spacer segment is left as a cohesive end.

On the other hand, in the case of bacteria, recombination will occur between their two loxP sites to cause insertion or deletion reaction (FIG. 2). If insertion reaction can be caused in mammalian cells, any gene can be inserted subsequently, thus resulting in a significantly wider range of applications. Since mammalian cells have large nuclei, circular DNA whose loxP has been deleted will diffuse and little insertion reaction is observed.

For this reason, the inventors of the present invention have attempted to introduce a mutation into a loxP sequence to cause insertion reaction such that once a gene has been inserted into the genome, the inserted gene cannot be deleted (i.e., cannot be eliminated from the genome), and have designed several types of loxP mutants (lox66, lox71, lox511, lox2272) for this purpose (FIG. 2). These loxP mutants are known (WO01/005987, JP 2007-100 A).

Moreover, in the present invention, systems under the name Vlox can also be used. Vlox refers to a Vibrio sp.-derived recombination system, VCre-Vlox (Suzuki, E., Nakayama, M. VCre/VloxP and SCre/CloxP: new site-specific recombination systems for genome engineering. Nucleic Acid Res. 2011, 1-11), and Vlox43L, Vlox43R, Vlox2272 and so on are available for use (FIG. 2).

The nucleotide sequences of loxP and loxP mutants as well as Vlox systems are shown below (FIG. 2).

loxP:
(SEQ ID NO: 1)
ATAACTTCGTATAGCATACATTATACGAAGTTAT
lox71:
(SEQ ID NO: 2)
TACCGTTCGTATAGCATACATTATACGAAGTTAT
lox66:
(SEQ ID NO: 3)
ATAACTTCGTATAGCATACATTATACGAACGGTA
lox511:
(SEQ ID NO: 4)
ATAACTTCGTATAGTATACATTATACGAAGTTAT
lox2272:
(SEQ ID NO: 5)
ATAACTTCGTATAGGATACTTTATACGAAGTTAT
Vlox:
(SEQ ID NO: 6)
TCAATTTCTGAGAACTGTCATTCTCGGAAATTGA
Vlox43L:
(SEQ ID NO: 7)
CGTGATTCTGAGAACTGTCATTCTCGGAAATTGA
Vlox43R:
(SEQ ID NO: 8)
TCAATTTCTGAGAACTGTCATTCTCGGAATACCT
Vlox2272:
(SEQ ID NO: 9)
TCAATTTCTGAGAAGTGTCTTTCTCGGAAATTGA

Further, in the present invention, the Dre/rox system can be used.

Dre refers to D6 site-specific DNA recombinase, which is an enzyme capable of recognizing the sequence of the rox site shown below (Sauer, B. and McDermott, Nucic Acid. Res. 32: 6086-6095, 2004). A recombination system based on this recombinase and the rox recognition sequence is referred to as the Dre/rox system. This system is closely related to the Cre-lox system although they differ in their DNA recognition specificity.

The nucleotide sequences of lox and rox are shown below.

rox:
(SEQ ID NO: 10)
5′-TAACTTTAAATAATGCCAATTATTTAAAGTTA-3′
(SEQ ID NO: 11)
3′-ATTGAAATTTATTACGGTTAATAAATTTCAAT-5′
lox:
(SEQ ID NO: 12)
5′-ATAACTTCGTATAATGTATGCTATACGAAGTTAT-3′
(SEQ ID NO: 13)
3′-TATTGAAGCATATTACATACGATATGCTTCAATA-5′

As described above, the present invention aims to establish mice with human normal tissues (e.g., human liver tissue), and further aims to establish model mice for tissue diseases (e.g., liver disease). For this purpose, in the present invention, ES cells are genetically engineered to ensure that a toxin is expressed in the cytoplasm of mouse liver cells to induce cell death in the mouse liver cells. Moreover, for the reason that human liver cells should be transplanted and grown to create a mouse with a human normal liver, the mouse growth hormone gene in ES cells is replaced with the human growth hormone gene. In addition, for analysis of functions such as drug metabolism, mouse drug-metabolizing enzyme genes are replaced with human drug-metabolizing enzyme genes.

A mouse introduced with liver cell death loses liver functions. Thus, this mouse not only can be used as a liver injury model, but can also be used to obtain a mouse with a humanized liver upon transplantation of human normal liver cells.

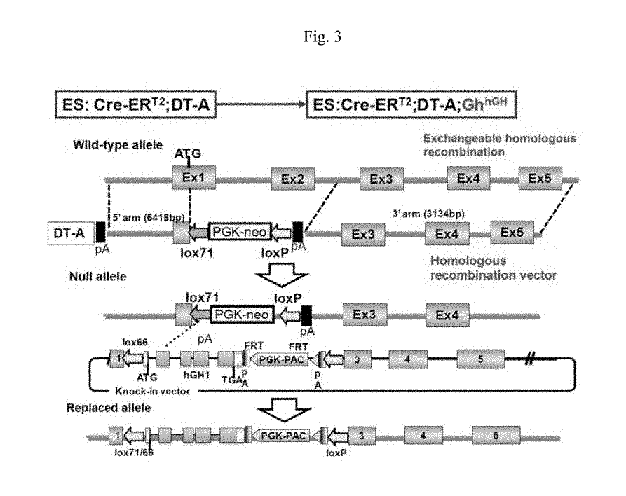

FIG. 3 shows a scheme for construction of a homologous recombination vector for replacement of the mouse growth hormone (GH) gene with the human GH gene.

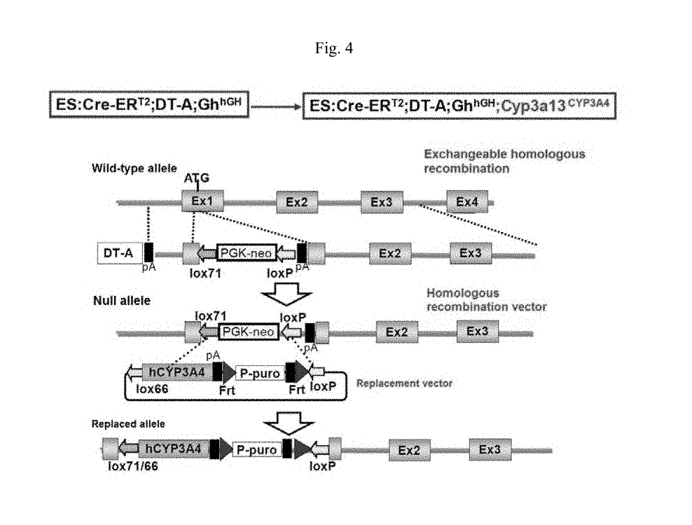

Likewise, FIG. 4 shows a scheme for construction of a homologous recombination vector for replacement of the Cyp gene, a drug-metabolizing enzyme gene, with the human Cyp gene.

Replacement of mouse genes with the above human genes can be accomplished in accordance with the gene trapping method described in WO01/005987. For example, two-step gene trapping may be conducted using a vector prepared as described above.

The first step is a commonly used gene trapping method. In this commonly used gene trapping, the above trapping vector is introduced into ES cells to trap an endogenous gene inherently present in the ES cells. As a result, the endogenous gene in the ES cells is disrupted. Then, a human gene is ligated downstream of the lox sequence (e.g., lox66) on a plasmid (replacement vector), followed by the second step of gene trapping (FIGS. 3 and 4).

In the second step of gene trapping, the human gene (e.g., hGH, hCyp) ligated downstream of lox66 is introduced into the ES cells. As a result, the lox71 site in the trapping vector introduced during the first step causes recombination with lox66 in the vector introduced during the second step, whereby a modified gene containing a cassette composed of “(lox71/66)-(human gene)-(loxP)” can be introduced. It should be noted that the puromycin resistance gene (puro) may be ligated between the human gene and loxP.

According to this method, endogenous mouse genes can be replaced with human genes. FIGS. 3 and 4 show the replaced alleles.

In FIGS. 3 and 4, Ex1, Ex2, Ex3 and Ex4 represent exons 1 to 4, respectively, in the mouse growth hormone gene or the mouse Cyp3a13 gene, pA represents a polyA sequence, Frt represents a FLP recognition site, PGK-neo represents the neomycin resistance gene ligated with PGK promoter, and P-puro represents the puromycin resistance gene ligated with PGK promoter.

In the case of other organs, the same strategy as described above for liver humanization is also used, as long as they are organs capable of serving as targets of organ transplantation. Namely, a gene may be prepared to comprise Cre-ER^{T2} ligated to an organ- or tissue-specific promoter, and this gene may be introduced into ES cells of HHB origin together with a vector such as CAG-lox-EGFP-lox-DT-A (construct 1 described later).

For example, to prepare a mouse with a humanized heart, a gene may be prepared to comprise Cre-ER^{T2} ligated to a cardiac-specific promoter, i.e., αMHC (α-myosin heavy chain) promoter, and this gene (MC: α-myosin heavy chain-Cre-ER^{T2}) may be introduced into HHB ES cells together with CAG-loxP-EGFP-loxP-DT-A (CD), whereby an HHB:MCCD mouse can be prepared. Once a fetal heart of this mouse has been transplanted with human cardiomyocytes and then administered with tamoxifen, mouse cardiomyocytes will be killed to give a mouse with a humanized heart where only the human cardiomyocytes have survived. Since human cardiomyocytes can be prevented from eliciting rejection reactions when they have been transplanted at the fetal stage, recipient mice after being grown up may be administered with tamoxifen or subjected to coronary artery ligature to thereby prepare a myocardial infarction model, into which human cardiomyocytes may then be transplanted.

5. Preparation of Chimeric Mouse

Preparation of a chimeric mouse can be accomplished in a standard manner.

First, the above established ES cells or gene-introduced or -replaced ES cells are allowed to aggregate with an eight-cell embryo or are injected into a blastocyst. The thus prepared embryo is referred to as a chimeric embryo, and this chimeric embryo is transplanted into the uterus of a pseudopregnant foster mother, which is then allowed to give birth, thereby preparing a chimeric mouse.

For example, to prepare a chimeric embryo, a female mouse treated with a hormone drug for superovulation may first be crossed with a male mouse. Then, after a given number of days have passed, an embryo at early development stage may be collected from the uterine tube or uterus. The collected embryo may be aggregated or injected with ES cells to prepare a chimeric embryo.

The term “embryo” as used herein is intended to mean an individual at any stage from fertilization to birth during ontogeny, including a two-cell embryo, a four-cell embryo, an eight-cell embryo, a morula stage embryo, a blastocyst and so on. An embryo at early development stage can be collected from the uterine tube or uterus at 2.5 days after fertilization for use as an eight-cell embryo and at 3.5 days after fertilization for use as a blastocyst.

For preparation of an aggregate using ES cells and an embryo, known techniques such as the microinjection method, the aggregation method and so on can be used. The term “aggregate” is intended to mean an aggregate formed from ES cells and an embryo gathering together in the same space, and includes both cases where ES cells are injected into an embryo and where an embryo is dissociated into separate cells and aggregated with ES cells.

In the case of using the microinjection method, the collected embryo may be injected with ES cells to prepare a cell aggregate. Alternatively, in the case of using the aggregation method, ES cells may be aggregated by being sprinkled over a normal embryo whose zona pellucida has been removed.

On the other hand, a pseudopregnant female mouse for use as a foster mother can be obtained from a female mouse with normal sexual cycle by crossing with a male mouse castrated by vasoligation or other techniques. The thus created pseudopregnant mouse may be transplanted in the uterus with a chimeric embryo prepared as described above and then allowed to give birth, thereby preparing a chimeric mouse.

From among the thus prepared chimeric mice, a male mouse derived from the ES cell-transplanted embryo is selected. After the selected male chimeric mouse has been matured, this mouse may be crossed with a pure-line female mouse. Then, if the coat color of the ES cell-derived mouse appears in the born pups, it can be confirmed that pluripotent stem cells have been introduced into the germ line of the chimeric mouse.

6. Preparation of Humanized Mouse

(1) Preparation of Genetically Modified Mouse which is Most Suitable for Humanization

Such a transgenic mouse (i.e., genetically modified mouse) established by using gene-introduced or -replaced ES cells serves as a base for establishment of a mouse with a 100% humanized organ (e.g., liver), as described later.

Since it has been clarified that rejection reactions can be avoided when human liver cells are transplanted via the fetal yolk sac vessel, ES cells from normal and HHB mice are used.

(i) Normal Mouse:

All mice can be applied as long as they are inbred mice already established. The present invention provides a method for creating a mouse with a humanized organ, characterized in that cells derived from the corresponding human organ are transplanted into a normal mouse fetus via the yolk sac vessel.

### (ii) HHB Mouse:

In the present invention, not only the above inbred mice, but also an HHB mouse can be used. This HHB mouse is a mouse having the genetic background of C57BL/6 mice introduced with deficiencies in the H2-D and β2-microglobulin genes and further modified to have the HHD gene.

(2) Preparation of a Liver Injury Model Mouse

For preparation of a liver injury model mouse, an antiestrogen may be administered to cause toxin expression to thereby eliminate (kill) mouse liver cells, thus obtaining an injury model mouse losing its liver functions.

To kill mouse liver cells or to express Cre-ER^{T2} in the cytoplasm of mouse liver cells, the following constructs 1 and 2 are prepared. Cre-ER^{T2} is a vector carrying the Cre recombinase gene ligated to a mutated estrogen receptor gene modified to prevent binding with estrogen produced in the mammalian body.

Construct 1:

CAG-ATG-lox-EGFP-lox-DT-A

Construct 2:

SAP-Cre-ER^{T2}

Construct 1 is composed of (i) ATG, (ii) EGFP flanked by lox sites and (iii) DT-A (diphtheria toxin fragment A), which are ligated immediately downstream of the CAG promoter.

This construct is designed to ensure in-frame ligation between the initiation codon in EGFP and ATG located upstream of lox. This construct is also designed to remove the initiation codon in DT-A and to ensure in-frame ligation with ATG located upstream of rox.

Construct 2 is composed of Cre-ER^{T2} ligated immediately downstream of the promoter for liver cell-specific serum amyloid P component (SAP).

When these constructs 1 and 2 are co-introduced into the ES cells of the present invention, site-specific recombination will occur after tamoxifen administration, and diphtheria toxin will be expressed in a manner specific to liver cells, whereby cell death can be induced.

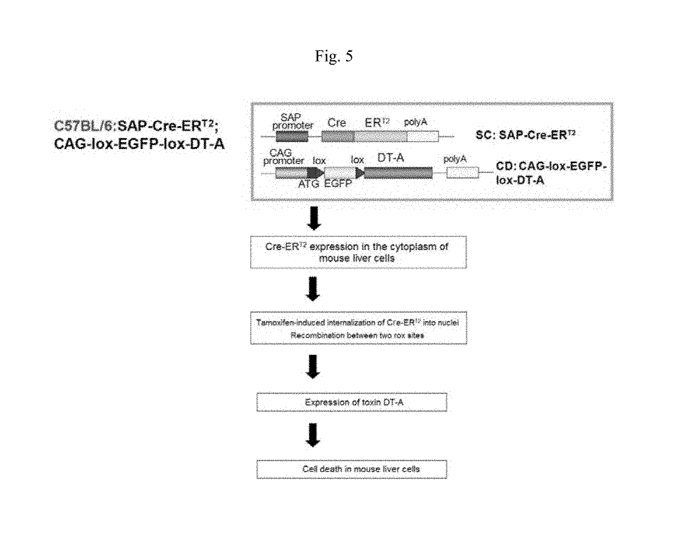

Namely, as a non-steroidal antiestrogen, for example, tamoxifen is a substance which has antitumor activity as a result of binding to the estrogen receptor in a manner competitive with estrogen to thereby exert an anti-estrogenic effect. When Dre-ER^{T2}-expressing humanized mice are administered with tamoxifen, Dre-ER^{T2} will be transferred into their nuclei by the action of tamoxifen. Recombination between two lox sites will occur to allow the promoter for the diphtheria toxin gene to function. As a result, toxin DT-A will be expressed to kill mouse liver cells (FIG. 5).

Tamoxifen may be administered at any frequency and for any period as long as liver cells can be killed, although it is administered as follows, by way of example.

Starting at 18.5 days of embryonic age, tamoxifen is given by being mixed into a mash feed at a ratio of 0.1 g/200 g feed. After 2 days, the mice give birth and are administered with a normal feed for 3 days. Subsequently, the mice are fed at the same concentration for 1 week and then administered with the normal feed for 3 days. Thereafter, the mice are continuously fed at the same concentration.

(3) Preparation of Humanized Mouse Whose Liver Cells are Replaced with Human Liver Cells

For preparation of a mouse whose liver cells are replaced with human liver cells, mouse liver cells may be eliminated by antiestrogen administration, as described above, and also human liver cells may be transplanted into a mouse, thus obtaining a humanized mouse whose liver cells are replaced with human liver cells.

Establishment of a mouse with a human normal liver is necessary to maintain liver functions over a long period of time and confirm the safety.

(i) Preparation of ES Cells in which the Mouse Growth Hormone Gene is Replaced with the Corresponding Human Gene

To ensure the growth of the transplanted human liver cells, the mouse growth hormone gene is replaced with the corresponding human gene at the stage of ES cells.

More specifically, gene replacement in ES cells may be accomplished in two steps, as described above.

In the first step, normal cells engineered to have SAP-Cre-ER^{T2} and CAG-lox-EGFP-lox-DT-A (hereinafter referred to as ES:SAP-Cre-ER^{T2}; CAG-lox-EGFP-lox-DT-A (ES:SCCD)) and HHB ES cells engineered to have SAP-Cre-ER^{T2} and CAG-lox-EGFP-lox-DT-A (hereinafter referred to as HHB ES:SAP-Cre-ER^{T2}; CAG-lox-EGFP-lox-DT-A (HHB ES:SCCD) are used for homologous recombination to disrupt the mouse growth hormone gene at its initiation codon and also establish ES cells) (ES:SCCD; Gh^{neo}) or HHB ES cells (HHB ES:SCCD; Gh^{neo}), each carrying lox71-PGK-neo-loxP integrated into this site.

In the second step, these ES cells and a replacement vector may be used to establish ES cells (ES:SCCD; Gh^{hGH} or HHB ES:SCCD; Gh^{hGH}) carrying human growth hormone gene cDNA in place of the neo gene.

The thus established ES cells may be used to obtain a mouse producing human growth hormone.

(ii) Elimination of Mouse Liver Cells and Undifferentiated Liver Cells

The administration frequency and administration period of tamoxifen are the same as described above.

(iii) Preparation of Human Liver Cells to be Transplanted

Human liver cells to be transplanted may be induced from iPS cells.

To obtain human liver cells, efficient techniques can be established for induction of endodermal and hepatic differentiation from human iPS cells with the use of supporting cells or an extracellular matrix.

iPS cells can be induced from somatic cells upon introduction of genes encoding 3 to 6 transcription factors (nucleus initialization factors) including members of Oct, Sox, Klf, Myc, Nanog, Lin and other families (Takahashi, K., et al. Induction of pluripotent stem cells from fibroblast cultures. Nat. Protoc. 2, 3081-9 (2007); Fusaki N, Ban H, Nishiyama A, Saeki K, Hasegawa M. Efficient induction of transgene-free human pluripotent stem cells using a vector based on Sendai virus, an RNA virus that does not integrate into the host genome. Proc Jpn Acad Ser B Phys Biol Sci. 2009; 85(8):348-62).

Members of the Oct family include, for example, Oct3/4, Oct1A, Oct6 and so on, with Oct3/4 being preferred.

Members of the Sox (SRY-related HMG box) family include, for example, Sox1, Sox2, Sox3, Sox7, Sox15 and so on, with Sox2 being preferred.

Members of the Klf (Kruppel-like factor) family include, for example, Klf1, Klf2, Klf4, Klf5 and so on, with Klf4 being preferred.

Members of the Myc family include c-Myc, N-Myc, L-Myc and so on, with c-Myc being preferred.

Nanog is a homeobox protein that is most highly expressed in the inner cell mass of blastocysts, but not expressed in differentiated cells.

Members of the Lin family include, for example, Lin28 which is used as a marker for undifferentiated human ES cells.

More specifically, preferred transcription factors are a combination of Oct3/4, Sox2, Klf4 and c-Myc (Takahashi, K. and Yamanaka, S., Cell 126, 663-676 (2006)), but it is also possible to use a combination of Oct3/4, Sox2 and Klf4 or a combination of Oct3/4, Sox2, Klf4 and L-Myc.

Examples of somatic cells include skin cells, liver cells, fibroblasts, lymphocytes and so on.

Techniques for gene transfer into somatic cells include, but are not limited to, lipofection, electroporation, microinjection, virus vector-mediated transfer, etc. Virus vectors used for this purpose include, for example, retrovirus vectors, lentivirus vectors, adenovirus vectors, adeno-associated virus vectors, Sendai virus and so on. It is also possible to use commercially available vectors, as exemplified by Sendai virus (DNAVEC).

In the case of using vectors, a gene to be introduced may also be operably linked to a regulatory sequence (e.g., a promoter, an enhancer) to ensure its expression. Examples of such a promoter include CMV promoter, RSV promoter, SV40 promoter and so on. These vectors may further comprise a positive selection marker such as a drug resistance gene (e.g., puromycin resistance gene, neomycin resistance gene, ampicillin resistance gene, hygromycin resistance gene), a negative selection marker (e.g., diphtheria toxin A fragment gene or thymidine kinase gene), IRES (internal ribosome entry site), a terminator, a replication origin and so on.

Somatic cells (e.g., 0.5×10⁴ to 5×10⁶ cells/100 mm dish) are transfected with a vector comprising the above nucleus initialization factors and cultured at about 37° C. on MEF feeders or under feeder-free conditions, whereby iPS cells are induced after about 1 to 4 weeks.

Examples of a medium include GMEM medium (Glasgow's Minimal Essential Medium), DMEM (Dulbecco's Modified Eagle's Medium), RPMI 1640 medium, OPTI-MEMI medium and so on. The culture medium may be supplemented as appropriate with an additional ingredient(s) selected from KSR (knockout serum replacement), fetal bovine serum (FBS), activin-A, basic fibroblast growth factor (bFGF), retinoic acid, dexamethasone, β-mercaptoethanol, nonessential amino acids, glutamic acid, sodium pyruvate and antibiotics (e.g., penicillin, streptomycin), etc.

Culture may be continued for a given period of time, followed by incubation in a medium containing EDTA or collagenase IV to collect the cells, as in the case of ES cell culture. Under feeder-free conditions, the cells may be cultured on Matrigel-coated plates in an MEF-conditioned medium.

It is usual to induce differentiation from iPS cells into human liver cells via three steps. In principle, these three steps are as follows:

(a) induction from pluripotent stem cells into the endodermal lineage,

(b) induction from the endodermal lineage into immature liver cells, and

(c) induction from the immature liver cells into mature liver cells.

In the above step (a), activin A and Wnt signals appear to be important. Likewise, FGF and BMP appear to be important in the step (b), while hepatocyte growth factor, oncostatin and dexamethasone appear to be important in the step (c).

However, in the above steps (b) and (c), these important factors may be replaced as appropriate with DMSO and retinoic acid, FGF4 and hydrocortisone and so on.

Transplantation of human liver cells may be conducted between 15.5 and 17.5 days of embryonic age or in adult mice at around 8 weeks after birth.

The number of human liver cells to be transplanted is preferably 10⁵ to 10⁶.

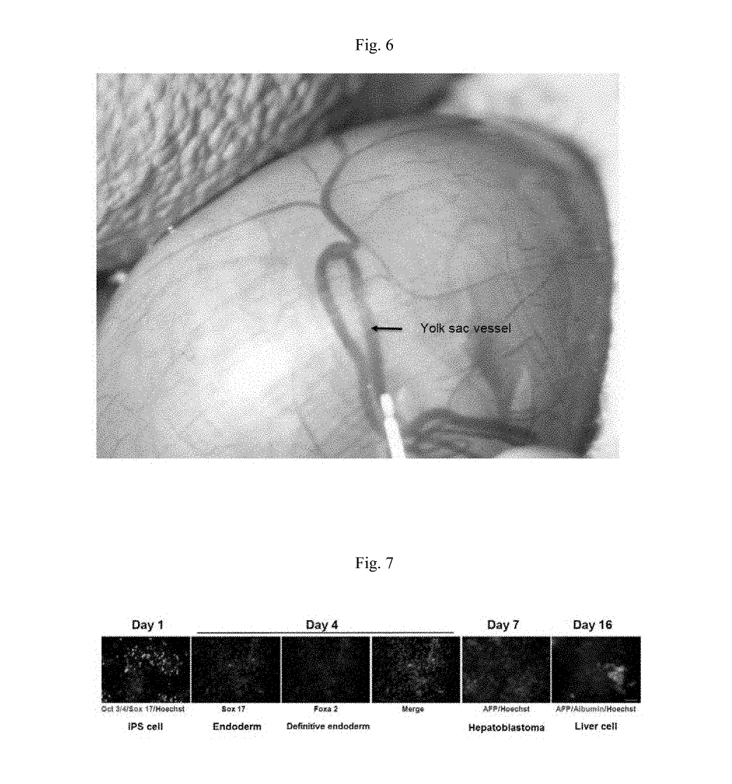

As to the route for transplantation of human liver cells, the cells may be transplanted through injection into the yolk sac vessel in the case of embryos (FIG. 6). In the case of adult mice, the cells may be injected into the spleen.

(iv) Growth of Human Liver Cells

The mouse established using ES cells in which the mouse growth hormone gene has been replaced with the human growth hormone gene is able to produce human growth hormone. This human growth hormone acts on the transplanted human liver cells to promote their growth, whereby it is possible to establish a humanized liver mouse with a human liver of normal size.

To confirm that all (100%) of the mouse liver cells are replaced with human liver cells, i.e., to confirm the absence of mouse liver cells, genes which are expressed in the mouse liver may be analyzed for their expression by RT-PCR or other techniques.

(4) Evaluation of Humanized Liver Mouse

To confirm that the liver has been humanized, the following characteristics may be tested either alone or in appropriate combination.

(i) Verification of Liver Functions

Characteristics to be tested for verification of liver functions include, for example, those listed below. The test period is not limited in any way, but it is preferably one year or longer.

Proteins: total protein, ALB, TTT, ZTT, CRP, Haptoglobin, C3, C4

Non-protein nitrogen component: total bilirubin, direct bilirubin

Carbohydrate: glucose

Lipid: triglyceride, total cholesterol, HDL-cholesterol, LDL-cholesterol, ApoAI, ApoCII

Enzyme: lactate dehydrogenase (LDH), aspartate aminotransferase (AST (GOT)), alanine aminotransferase (ALT (GPT)), γ-glutamyltransferase (GGT), creatine kinase (CK), alkaline phosphatase (AP), amylase (AML)

Others: calcium, Fe, inorganic phosphate

ICG test: Indocyanine green (ICG) is intravenously administered and the ICG concentration in blood is measured over time to test the dye excretory function of the liver. ICG is bound to lipoproteins in blood and transported to the liver, and is taken up into liver cells during passing through sinusoids and then excreted into bile without being conjugated. Thus, the functions of the liver can be analyzed as a whole organ, but not as liver cells.

CT test: Morphological changes in the liver are tested.

(ii) Drug Metabolism

PCR array techniques are used to analyze the drug metabolism-related enzymes listed below.

Cytochrome P450: CYP11A1, CYP11B1, CYP11B2, CYP17A1, CYP19A1, CYP1A1, CYP1A2, CYP1B1, CYP21A2, CYP24A1, CYP26A1, CYP26B1, CYP26C1, CYP27A1, CYP27B1, CYP2A13, CYP2R1, CYP2S1, CYP2B6, CYP2C18, CYP2C19, CYP2C8, CYP2C9, CYP2D6, CYP2E1, CYP2F1, CYP2W1, CYP3A4, CYP3A11, CYP3A13, CYP3A43, CYP3A5, CYP3A7, CYP3A25, CYP3A41, CYP4A11, CYP4A22, CYP4B1, CYP4F11, CYP4F12, CYP4F2, CYP4F3, CYP4F8, CYP7A1, CYP7B1, CYP8B1.

In the present invention, preferred is at least one selected from CYP3A11, CYP3A13, CYP3A25 and CYP3A41.

It should be noted that endogenous drug metabolism-related enzyme genes present in mouse cells are expressed with small alphabets except for their head alphabet. By way of example, the human “CYP11A1” gene is expressed as “Cyp11a1” for the corresponding mouse gene, while the human “CYP3A11” gene is expressed as “Cyp3a11” for the corresponding mouse gene.

Alcohol dehydrogenase: ADH1A, ADH1B, ADH1C, ADH4, ADH5, ADH6, ADH7, DHRS2, HSD17B10 (HADH2).

Esterase: AADAC, CEL, ESD, GZMA, GZMB, UCHL1, UCHL3.

Aldehyde dehydrogenase: ALDH1A 1, ALDH1A2, ALDH1A3, ALDH1B1, ALDH2, ALDH3A1, ALDH3A2, ALDH3B1, ALDH3B2, ALDH4A1, ALDH5A1, ALDH6A1, ALDH7A1, ALDH8A1, ALDH9A1.

Flavin-containing monooxygenase: FMO1, FMO2, FMO3, FMO4, FMO5.

Monoamine oxygenase: MAOA, MAOB.

Prostaglandin-endoperoxide synthase: PTGS1, PTGS2.

Xanthine dehydrogenase: XDH.

Dihydropyrimidine dehydrogenase: DPYD.

(iii) In Vitro Verification of Liver Cell Functions

Since liver cells are of endodermal origin, test cells may be examined for time-dependent expression of genes which are expressed in the endodermal lineage and liver cells, accumulation of glycogen, expression of cytochrome enzymes and so on to thereby verify whether the test cells have human liver functions.

The time-dependent expression of genes which are expressed in the endodermal lineage and liver cells may be verified for Oct3/4, T, Gsc, Mix11, Foxa2, Hex, Hnf4a, Hnf6, Afp, Alb, Ttr, αAT, etc. Techniques for their verification include, for example, commonly used Northern blotting, RT-PCR and Western blotting.

The secretory ability of liver cells may be verified by measuring ALB, transferrin, alpha 1-antitrypsin and fibrinogen for their concentrations in the culture solution. Techniques for their verification include, for example, commonly used Western blotting or EIA (enzyme-immuno assay).

The accumulation of glycogen may be verified by PAS (periodic acid-Schiff) staining. Periodic acid selectively oxidizes glucose residues to generate aldehydes, causing a color change to red purple by the action of Schiff's reagent.

The expression of cytochrome enzymes may be verified by analysis of five major enzymes, i.e., CYP3A4, CYP1A2, CYP2C9, CYP2C19 and CYP2D6. Techniques for their verification include, for example, commonly used Northern blotting, RT-PCR and Western blotting.

(5) Preparation of Liver Disease Model Mouse Whose Liver Cells are Replaced with Human Patient-Derived Liver Cells

The mouse of the present invention may be transplanted with human patient-derived liver cells and also administered with an antiestrogen to eliminate liver cells originating from the mouse, whereby a human liver disease model mouse can be obtained.

Establishment of a mouse with a human mutated liver is necessary for establishment of a disease model having the same symptoms as seen in human patients and for pathology analysis. Moreover, a model optimized for human diseases is established and can be used for development of a novel therapy used for a wide range of purposes.

### EXAMPLES

The present invention will be further described in more detail by way of the following examples, although the present invention is not limited to these examples. It should be noted that all applications for induction of liver cells from iPS cells, establishment of iPS cells from patients with human familial amyloid polyneuropathy or patients with human propionic acidemia, and transplantation experiments of the induced human liver cells into mice were approved by the ethical committee, the animal research committee, and the safety committee on recombinant DNA experiments of class 2.

### Example 1

Establishment of ES Cells

In this example, for establishment of a humanized optimal mouse most suitable for human liver cell transplantation, ES cell lines were established from HHB mouse embryos, and mouse strains thereof were also established.

(1) Establishment of HHB Mouse and ES Cell Lines Thereof

HHB mice were used for in vitro fertilization to obtain 33 blastocyst embryos, and the GSK3 inhibitor CHIR99021 and the MEK inhibitor PD0325901, which are considered to be effective for maintenance of the undifferentiated state of ES cells, were added to the medium (GMEM-KSR-2i medium) in an attempt to establish ES cell lines.

More specifically, HHB embryos were collected by in vitro fertilization. 33 blastocysts were cultured in KSOM medium for 4 days until they became blastocysts, and the embryos were transferred on a one-by-one basis to 48 wells (coated with gelatin alone). The medium used was KSR-GMEM-2i medium composed of G-MEM (Glasgow minimum essential medium) supplemented with 1× MEM nonessential amino acids, 0.1 mM β-mercaptoethanol, 1 mM sodium pyruvate, 1% fetal bovine serum (FBS) (Hyclone), 14% Knockout™ SR (KSR), 1100 uints/ml leukemia inhibitory factor (LIF), 2 μM PD0325901 and 3 μM CHIR99021. The culture period was set to 14 days, during which the medium was replaced twice. After 14 days to 18 days, subculture was conducted from wells with increased ICM to 24 wells containing feeder cells. Further, subculture was conducted sequentially in 12 wells, 6 wells and 6-cm dishes, finally establishing 21 lines of ES cells having no problem in growth rate and morphology.

(2) Preparation of Chimeric Mice Using HHB ES Cell Lines and Establishment of HHB Mouse Strains

Among the established ES lines, 12 cell lines were used to prepare chimeric mice by being aggregated with morula embryos obtained by crossing between B6 female and BDF1 male mice (Table 1).

Germ-line transmission was confirmed in 100% chimeras obtained from three ES lines (HHB-3, HHB-9 and HHB-10).

It should be noted that among the resulting ES cell lines, the 10th cell line was designated as “HHB10” and was internationally deposited under the Budapest Treaty on Jun. 17, 2015 (receipt date) with the National Institute of Technology and Evaluation, Patent Microorganisms Depositary (2-5-8 Kazusakamatari, Kisarazu-shi, Chiba 292-0818, Japan). Its Accession No. is “NITE ABP-02068.”

TABLE 1

| | Number of embryos | Number of pups | Number of 100% |
|---|---|---|---|
| Line No. | transplanted | born | chimeras |
| 2 | 75 | 3 | 2♂ |
| 3 | 75 | 12 | 6♂ |
| 4 | 75 | 8 | 4♂ |
| 5 | 50 | 0 | 0 |
| 6 | 75 | 5 | 3♂ |
| 7 | 75 | 3 | 3♂ |
| 8 | 50 | 3 | 0 |
| 9 | 75 | 14 | 9♂ |
| 10 | 75 | 16 | 11♂ |
| 11 | 75 | 11 | 8♀ |
| 12 | 75 | 2 | 0 |
| 13 | 75 | 0 | 0 |

### Example 2

Induction of Cell Death in Mouse Liver Cells

### (1) Preparation of Constructs for Induction of Cell Death in Mouse Liver Cells

For preparation of a genetically modified mouse capable of specifically causing death in liver cells, two constructs were prepared.

Construct 1 (CAG-ATG-lox-EGFP-lox-DT-A) is composed of ATG, EGFP flanked by lox sites and DT-A (diphtheria toxin fragment A), which are ligated immediately downstream of the CAG promoter.

This construct was designed to ensure in-frame ligation between the initiation codon in EGFP and ATG located upstream of rox. This construct was also designed to remove the initiation codon in DT-A and to ensure in-frame ligation with ATG located upstream of rox.

Construct 2 (SAP-CreER^{T2}) is composed of Cre-ER^{T2} ligated immediately downstream of the promoter for liver cell-specific serum amyloid P component (SAP). In addition, the puromycin resistance gene is ligated upstream of the SAP promoter.

Detailed procedures are as shown below.

(1-1) Construct 1

Construct 1 was prepared in the following manner.
(i) p6SEAZ and pSP-rox2 were treated with restriction enzymes PstI and KpnI, respectively, and then blunt-ended with T4 Polymerase (TaKaRa). Subsequently, they were treated with a restriction enzyme EcoRI and ligated to each other to prepare pSP-lox-EGFP-lox.
(ii) pSP-lox-EGFP-lox and pBSK-atg-rox2 (synthetic DNA, Biomatik) were treated with restriction enzymes EcoRI and SmaI, and then ligated to each other to prepare pBSK-atg-lox-EGFP-lox.
(iii) pBSK-atg-lox-EGFP-lox and P71hAXC-DT were treated with restriction enzymes BamHI and PstI, and then ligated to each other to prepare pBSK-atg-lox-EGFP-lox-DT-A.
(iv) pCAGGS-EGFP and pBSK-atg-lox-EGFP-lox-DT-A were treated with restriction enzymes KpnI and SpeI, respectively, and then blunt-ended with T4 Polymerase (TaKaRa). Subsequently, they were treated with a restriction enzyme Hind III and then ligated to each other to prepare CAG-atg-lox-EGFP-lox-DT-A.

(1-2) Construct 2

Construct 2 was prepared in the following manner.
(i) pkSAP-CrePP was used as a template in PCR to amplify a region covering from the initiation codon to the last codon before the stop codon. The reverse primer was provided with a BamHI site.

PCR kit: TaKaRa Ex Taq
Fw Primer:
(SEQ ID NO: 14)
CCATGGCCCCCAAGAAGAAAA
Re Primer:
(SEQ ID NO: 15)
CGGGATCCATGAGCCTGCTGTT

pGEM-T Easy Vector and the above PCR product were ligated to each other to prepare T easy-Dre.
(ii) pkSAP-CrePP and T easy-Cre were treated with restriction enzymes SalI and EcoRI, and then ligated to each other to prepare T Easy SAP.
(iii) The above T Easy Cre and T easy-SAP were treated with restriction enzymes SacII and NotI, and then ligated to each other to prepare T easy-SAP-Cre.
(iv) T Easy-SAP-Cre and pkSA-CremER^{T2}PP were used and treated with restriction enzymes BamHI and NotI, and then ligated to each other to prepare T easy-SAP-CremER^{T2}.
(v) pkSAP-CrePP and T easy-SAP-CremER^{T2} were treated with restriction enzymes SalI and NotI, and then ligated to each other to prepare pKSAP-CreER^{T2}.
(vi) pKSAP-CreER^{T2} and pFPacpaF2 were treated with restriction enzymes SpeI and KpnI, respectively, and then blunt-ended with T4 polymerase (TaKaRa). Subsequently, pKSAP-CreER^{T2} and pFPacpaF2 were treated with restriction enzymes SalI and XhoI, respectively, and then ligated to each other to prepare Puro-SAP-CreER^{T2}.

(2) Introduction of Estrogen Receptor Gene and Diphtheria Toxin Gene into ES Cells

Conditions were studied to ensure efficient expression of human genes upon insertion (Li, Z. et al., Transgenic Res. 20:191-200, 2011. DOI 10.1007/s11248-010-9389-22).

The presence or absence of a PGK-puromycin cassette and IRES was analyzed to determine which combination would achieve the highest expression efficiency.

Prior to the analysis, a homologous recombination vector was used to disrupt the first exon of the mouse transthyretin (Ttr) gene in a standard manner (Zhao, G., Li, Z., Araki, K., Haruna, K., Yamaguchi, K., Araki, M., Takeya, M., Ando, Y. and Yamamura, K. Inconsistency between hepatic expression and serum concentration of transthyretin in mice humanized at the transthyretin locus. Genes Cells 13: 1257-1268, 2008). During this treatment, ATG in the first exon was disrupted, resulting in a target recombinant clone carrying lox71-PGK-beta-geo-loxP-polyA-lox2272 integrated into this site.

Then, two types of replacement vectors were prepared. Replacement vector 1 comprises lox66-hTTR cDNA-polyA-Frt-PGK-puro-Frt-loxP, while replacement vector comprises lox66-IRES-hTTR cDNA-polyA-Frt-PGK-puro-Frt-loxP. These replacement vectors were each introduced together with a Cre expression vector into the target recombinant clone by electroporation.

As a result, the following two clones were obtained: lox71/66-hTTR cDNA-polyA-Frt-PGK-puro-Frt-loxP (abbreviated as I(−)P(+)) and lox71/66-IRES-hTTR cDNA-polyA-Frt-PGK-puro-Frt-loxP (abbreviated as I(+)P(+)). These two clones each have PGK-puro, but I(−)P(+) has no IRES.

Into these two clones, CAG-FLP was introduced by electroporation and PGK-puro between Frt sites was deleted to prepare I(−)P(−) and I(+)P(−) clones.

Mice were prepared from these four ES clones and subjected to expression analysis, indicating that I(−)P(+) showed the highest expression, followed by I(−)P(−), I(+)P(+) and I(+)P(−) in decreasing order. Moreover, in the case of I(−)P(+), human TTR (transthyretin) expression in the liver was found to be substantially equal to the expression levels of mouse Ttr (transthyretin) in control mice.

As a result, a combination of the presence of PGK-puromycin and the absence of IRES was found to achieve the highest expression efficiency for the inserted human gene.

### Example 3

Replacement with Human Growth Hormone Gene

Prior to the experiment, a homologous recombination vector was used to disrupt the first and second exons of the mouse growth hormone (Gh) gene in a standard manner as in the case of Example 2. During this treatment, ATG in the first exon was disrupted, resulting in a target recombinant clone carrying lox71-PGK-beta-geo-loxP-polyA-lox2272 integrated into this site. Then, a replacement vector was prepared. The replacement vector comprises lox66-genomic hGH gene-polyA-Frt-PGK-puro-Frt-loxP. This replacement vector was introduced together with a Cre expression vector into the target recombinant clone by electroporation.

As a result, an ES clone in which the mouse Gh gene was replaced with the human GH gene was obtained.

### Example 4

Replacement with Human Drug-Metabolizing Enzyme Gene

Prior to the experiment, a homologous recombination vector was used to disrupt the first exon of the mouse Cyp3a13 gene in a standard manner. During this treatment, ATG in the first exon was disrupted, resulting in a target recombinant clone carrying lox71-PGK-beta-geo-loxP-polyA-lox2272 integrated into this site. Then, a replacement vector was prepared. The replacement vector comprises lox66-hCYP3A4 cDNA-polyA-Frt-PGK-puro-Frt-loxP. This replacement vector was introduced together with a Cre expression vector into the target recombinant clone by electroporation.

As a result, an ES clone in which the mouse Cyp3a13 gene was replaced with the human CYP3A4 gene was obtained.

### Example 5

Preparation of Mouse Whose Liver is Humanized

Techniques to induce differentiation from human iPS cells into human liver cells were substantially established, and constructs for induction of cell death in mouse liver cells were also prepared.

(1) Induction of Differentiation from Human iPS Cells into Liver Cells

Efficient techniques were constructed for induction of endodermal and hepatic differentiation from human iPS cells.

To cause differentiation from iPS cells into human liver cells, the iPS cells were cultured in a medium containing a Rock inhibitor from the first day to the second day. The cells were then cultured in DMEM medium from the third day to the fourth day. This DMEM medium contains the following: 4,500 mg/l glucose, activin A (100 ng/ml), CHIR99021 (3 μm) and bFGF (50 ng/ml).

The cells were then cultured in the presence of 4,500 mg/l glucose, 1 μm dexamethazone and 10 μm hepatocyte growth factor from the fourth day to the 13th day.

Finally, from the 15th day to the 30th day, the cells were cultured in DMEM medium containing the following: 10% KSR, 1 μm dexamethazone, 10 μm hepatocyte growth factor and oncostatin M (30 ng/mL).

(2) Study of Transplantation Techniques for iPS-Derived Human Liver Cells

With the aim of establishing techniques for efficient introduction of iPS-derived liver cells into mouse livers, which are required for humanization of livers, a method was developed for introducing iPS-derived human liver cells through the yolk sac vessel present on the mouse fetal amniotic membrane at 16.5 or 17.5 days of embryonic age (FIG. 6).

The liver cells prepared in (1) above were used for transplantation.

In this culture method, iPS cells were induced to differentiate into Sox17-positive endoderm at the 4th day of culture, into AFP-positive immature liver cells at the 7th day of culture, and into ALBUMIN-positive mature liver cells at the 16th day of culture.

In addition, the liver cells showed no mouse gene expression when analyzed by RT-PCR with mouse specific primers, thus indicating that 100% of the liver cells were of human origin.

The liver cells were transplanted, and livers were excised from the mice at the 14th day and immunostained with anti-human cytokeratin 8/18 antibody, thus indicating that the human liver cells were confirmed to be engrafted. In addition, the same analysis was conducted after 4 weeks, indicating that the colony size of human liver cells was increased, and that the human liver cells were incorporated into hepatic lobule structures.

### Example 6

Establishment of Mutated Humanized Liver Mice

In this example, FAP and PA model mice were bred.

(1) Induction of Mutated Liver Cells from Human Patients

### (i) Familial Amyloid Polyneuropathy (FAP): Already Established

FAP is an autosomal dominant hereditary disease caused by a point mutation in the transthyretin (TTR) gene. For example, in FAP, a replacement of valine with methionine occurs at amino acid position 30 in the amino acid sequence of transthyretin (Val30Met). Fibroblasts taken from patients having this Val30Met mutation were used to establish iPS cells.

As a result, it was indicated that these iPS cells were able to be induced to differentiate into liver cells in the same manner as described previously.

(ii) Establishment of iPS Cells from Human Propionic Acidemia (PA) Patients

PA is an autosomal recessive hereditary disease caused by a defect in the propionyl CoA carboxylase (PCCA) gene. For example, in PA, a replacement of arginine with tryptophan occurs at position 52 in the amino acid sequence of PCCA (Arg52Trp). Fibroblasts taken from patients having this mutation were used to establish iPS cells. As a result, it was indicated that these iPS cells were able to be induced to differentiate into liver cells in the same manner as described previously.

(2) Establishment of Mutated Humanized Liver Mice (Model Mice for FAP and PA)

Mutated humanized liver mice may be established in the same manner as used to prepare a humanized liver mouse (i.e., a mouse prepared by transplantation of liver cells induced from normal human-derived iPS cells). Namely, the mouse of the present invention may be transplanted with liver cells induced to differentiate from iPS cells derived from FAP and PA patients to thereby establish the mutated humanized liver mice.

### INDUSTRIAL APPLICABILITY

The present invention provides ES cells derived from an HHB mouse. An embryo prepared using the ES cells of the present invention may be transplanted with human cells to thereby create a mouse in which an organ of interest (e.g., liver) is humanized, and this mouse can be used to examine human organ functions.

### Deposition Number

Microorganism is labeled as: “HHB10”

Accession No.: NITE ABP-02068

Initial deposit date (receipt date): Jun. 17, 2015

International Deposition Authority:
National Institute of Technology and Evaluation, Patent Microorganisms Depositary
2-5-8 Kazusakamatari, Kisarazu-shi, Chiba 292-0818, Japan

### Sequence Listing Free Text

SEQ ID NOs: 1 to 15: synthetic DNAs

1. An embryonic stem cell obtained from an embryo of a mouse engineered to replace all or some of domains in the mouse MHC class I molecule H2-D with domains from the human MHC class I molecule HLA-A by culture in the presence of a GSK3 inhibitor and an MEK inhibitor.

2. The embryonic stem cell according to claim 1, wherein the α1 domain, α2 domain of the H2-D molecule and β2 microglobulin are replaced with the α1 domain, α2 domain of the human HLA-A molecule and β2 microglobulin, respectively.

3. The embryonic stem cell according to claim 1, which is deposited under Accession No. NITE ABP-02068.

4. The embryonic stem cell according to claim 1, which is engineered to have the estrogen receptor gene and the diphtheria toxin gene.

5. The embryonic stem cell according to claim 4, wherein the endogenous growth hormone gene in the cell is replaced with that of human origin.

6. The embryonic stem cell according to claim 5, wherein an endogenous drug-metabolizing enzyme gene in the cell is further replaced with that of human origin.

7. The embryonic stem cell according to claim 6, wherein the endogenous drug-metabolizing enzyme gene in the cell is at least one selected from the group consisting of Cyp3a11, Cyp3a13, Cyp3a25 and Cyp3a41.

8. A mouse, which is created with the use of the embryonic stem cell according to claim 1.

9. A mouse, which is created with the use of the embryonic stem cell according to claim 4.

10. The mouse according to claim 9, which develops liver cell injury upon administration of an antiestrogen.

11. A mouse with a humanized liver, wherein the mouse according to claim 9 is transplanted with liver cells of human origin and also administered with an antiestrogen to eliminate liver cells originating from the mouse.

12. The mouse according to claim 11, wherein the liver cells of human origin are derived from a patient with a liver disease.

13. A human liver disease model mouse, which consists of the mouse according to claim 12.

14. A method for preparing an embryonic stem cell of mouse origin, which comprises culturing, in the presence of a GSK3 inhibitor and an MEK inhibitor, an embryo of a mouse engineered to replace all or some of domains in the mouse MHC class I molecule H2-D with domains from the human MHC class I molecule HLA-A.

15. The method according to claim 14, wherein the α1 domain, α2 domain of the H2-D molecule and β2 microglobulin are replaced with the α1 domain, α2 domain of the human HLA-A molecule and β2 microglobulin, respectively.

16. A method for creating a liver injury model mouse, which comprises administering an antiestrogen to the mouse according to claim 9.

17. A method for creating a mouse with a humanized liver, which comprises transplanting liver cells of human origin into the mouse according to claim 9 and also administering an antiestrogen to eliminate liver cells originating from the mouse.

18. The method according to claim 17, wherein the liver cells of human origin are derived from a patient with a liver disease.
